(19) United States
(12) Patent Application Publication
(10) Pub. No.: US 2005/0015019 A1
(43) Pub. Date: Jan. 20, 2005

(54) Sampling syringe unit, sampling device and sampling method for sampling blood or body fluid

(75) Inventors: Nobuaki Honda, Tokyo (JP); Tetsuya Miyagishi, Tokyo (JP); Hiroyuki Yoshida, Tokyo (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: Yamatake Corporation, Shibuya-ku (JP)

(21) Appl. No.: 10/623,254

(22) Filed: Jul. 18, 2003

(51) Int. Cl. A61B 5/00

(52) U.S. Cl. 600/576

(57) ABSTRACT

A sampling syringe unit includes a syringe base for defining a chamber in cooperation with a diaphragm, a needle protruding substantially perpendicularly from the syringe base and communicating with the chamber, and a protective member which, in a normal state, is elastically urged to be located more outward than the tip of the needle. A sampling device to which the sampling syringe unit is removably attached includes a first actuator for displacing the syringe base, and a second actuator for displacing the diaphragm and then releasing the displacing force to restore the diaphragm and thereby cause a suction pressure to be produced in the chamber.

### BACKGROUND OF THE INVENTION

1. Field of the Invention

The present invention relates to a sampling syringe unit, a sampling device and a sampling method suited for sampling a very small quantity of blood or body fluid.

2. Description of the Related Art

Recently, researches have been actively pursued on pTAS's (Micro Total Analysis Systems) for performing chemical analyses of DNA, blood components, etc. or measurements of blood sugar level. This type of pTAS requires only a very small quantity of blood or body fluid to be sampled from a patient (subject) and thus is advantageous in that the patient is relieved of pain and burden to a certain degree.

Meanwhile, Japanese Patent No. 3155523 discloses a sampling device which has a reservoir containing an ion conducting medium and enzyme and utilizes iontophoresis to sample a very small quantity of blood or body fluid. Also, Unexamined Japanese Patent Publication No. 2000-185034 discloses a sampling device provided with a minute hollow needle made of silicon and adapted to sample blood by using a diffuser type micropump driven by a piezoelectric device.

In the former device, however, an electrolyte for effecting iontophoresis remains in contact with the skin for a long time, and accordingly, there is a possibility that the patient feels itchy depending on his/her constitution. The latter device is inevitably increased in size because of its structure, namely, because a diaphragm is formed on a flat plate of silicon and a channel for sampling blood or the like is formed along the flat plate. Moreover, each time blood is sampled, the hollow needle needs to be manually stuck into the patient's skin, like a syringe.

### SUMMARY OF THE INVENTION

An object of the present invention is to provide a sampling syringe unit for sampling blood or body fluid, which unit can be attached to a patient (subject) when used, is simple in structure and easy to handle, and a sampling device and method using the sampling syringe unit.

To achieve the object, the present invention provides a sampling syringe unit which is removably attached to a sampling device and is disposed of after use. The sampling syringe unit comprises: (a) a syringe base having an elastically deformable diaphragm attached to one surface thereof such that a chamber is defined between the diaphragm and the syringe base; (b) a hollow tubular needle having an outer diameter of, for example, 0.1 mm or less, the needle protruding substantially perpendicularly from a central portion of a surface of the syringe base opposite to the surface on which the diaphragm is arranged, and communicating with the chamber; and (c) a protective member having an aperture for passing the needle therethrough, the protective member facing the surface of the syringe base from which the needle protrudes and movable toward and away from the syringe base, wherein in a normal state, the protective member is located more outward than a tip of the needle.

Preferably, the syringe base is made of a thin plastic plate having a recess formed in one surface thereof to define the chamber, for example, and a flat diaphragm is bonded to the surface of the plastic plate so as to cover the recess, thereby making the sampling syringe unit shaped like a thin flat plate. The protective member may be positioned more outward than the tip of the needle by making use of the elastic force of a spring or the repulsive force of a magnet, for example. Alternatively, the protective member may be positioned by using a hook mechanism that selectively engages with the syringe base. Further; electrodes for examining a substance sampled through the needle may be arranged in a communication passage connecting the chamber of the syringe base and the needle, particularly, in a portion of the communication passage extending parallel with the diaphragm.

A sampling device according to the present invention comprises: (d) a housing unit to which the sampling syringe unit is removably attached in such a manner that the surface of the syringe base on which the needle is arranged faces outward; (e) a first actuator arranged in the housing unit and driven, for example, by a plunger, for displacing the syringe base attached to the housing unit in a direction toward the surface of the syringe base on which the needle is arranged; and (f) a second actuator arranged in the housing unit and driven, for example, by a plunger separate from the above plunger in association with the first actuator, for displacing the diaphragm in a direction toward the surface of the syringe base on which the needle is arranged, and then releasing the displacing force to restore the diaphragm and thereby cause a suction pressure to be produced in the chamber.

Alternatively, the sampling device of the present invention may comprise: (d) a housing unit to which the sampling syringe unit is removably attached in such a manner that the surface of the syringe base on which the needle is arranged faces outward; (g) an actuator arranged in the housing unit, for displacing the syringe base of the sampling syringe unit attached to the housing unit in a direction toward the surface of the syringe base on which the needle is arranged; and (h) a deformation control member for regulating deformation of the diaphragm accompanying the displacement of the syringe base, for example, the deformation control member deforming the diaphragm as the syringe base is displaced, to cause a suction pressure to be produced in the chamber, and then maintaining a deformed state of the diaphragm.

Both of the above sampling devices may be provided with a controller, such as a timer, for controlling operation timing thereof.

A sampling method according to the present invention is characterized by attaching a plurality of sampling devices to a subject by using a belt or the like, and sequentially driving the sampling devices by the controller to sample blood or body fluid from the subject at different times.

### BRIEF DESCRIPTION OF THE DRAWINGS

FIG. 1 is a diagram illustrating a basic structure of a sampling syringe unit according to one embodiment of the present invention, as well as schematic configuration of a sampling device to which the sampling syringe unit is removably attached for sampling blood or body fluid;

FIG. 2 is a diagram illustrating how a diaphragm is operated to produce a suction force within the sampling syringe unit;

FIG. 3A is a diagram showing a modification of the sampling syringe unit;

FIG. 3B is a diagram showing a state in which a diaphragm of the sampling syringe unit of FIG. 3A is depressed;

FIG. 4 is a diagram illustrating an example of how the sampling devices are attached to the human arm;

FIG. 5A is a diagram illustrating the relationship between the sampling syringe unit and the sampling device;

FIG. 5B is a diagram showing a state in which the sampling syringe unit of FIG. 5A is attached to the sampling device;

FIG. 6A is a diagram showing an initial state of the sampling device shown in FIG. 5B;

FIG. 6B is a diagram showing a state in which a needle of the sampling device of FIG. 5B is stuck into the skin;

FIG. 6C is a diagram showing a state in which blood has been drawn into the sampling device of FIG. 5B;

FIG. 7A is a diagram schematically illustrating the structure of a sampling syringe unit and sampling device according to another embodiment of the present invention;

FIG. 7B is a diagram showing a state in which a needle of the sampling device of FIG. 7A is stuck into the skin;

FIG. 7C is a diagram showing a state in which blood has been drawn into the sampling device of FIG. 7A; and

FIG. 7D is a diagram showing a state of the sampling device of FIG. 7A after the blood is sampled.

### DETAILED DESCRIPTION OF THE INVENTION

A sampling syringe unit, a sampling device and a sampling method for sampling blood or body fluid according to embodiments of the present invention will be hereinafter described with reference to the drawings.

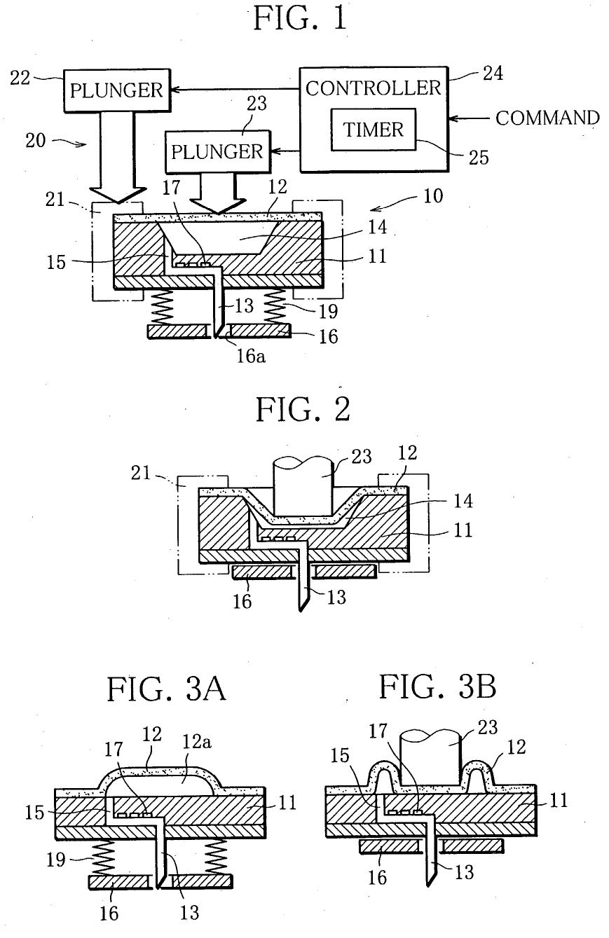

FIG. 1 illustrates a basic structure of a sampling syringe unit, along with schematic configuration of a sampling device to which the sampling syringe unit is removably attached for sampling blood or body fluid. The sampling syringe unit 10 comprises a syringe base 11, a diaphragm 12, and a needle 13. The syringe base 11 is made of a thin plastic plate, for example. The diaphragm 12 is made of an elastic material and is bonded to one surface of the syringe base 11 to define a suction chamber, described later, in cooperation with the syringe base 11. The needle 13 is a hollow tube made of stainless steel and protrudes from a central portion of the other or reverse surface of the syringe base 11 opposite to the diaphragm in a direction substantially perpendicular to the syringe base.

Specifically, the suction chamber with a predetermined capacity is defined by, for example, a truncated cone-shaped recess 14 formed in the surface of the syringe base 11 and the diaphragm 12 closing the opening of the recess 14. The needle 13 is obtained, for example, by drawing a tube of stainless steel into a hollow tube having an outer diameter of 0.1 mm or less and a length of about 1 to 2 mm. The needle 13 is attached to the reverse surface of the syringe base 11 in communication with the recess 14 through a communication passage 15 formed in the syringe base 11.

The syringe base 11 having the recess 14 and the communication passage 15 is obtained, for example, by bonding two plastic plates (parts) together each having a hole or groove of predetermined shape formed beforehand. Also, three electrodes 17, for example, a working electrode, a counter electrode and a reference electrode, all of platinum, are formed in the inner wall surface of the communication passage 15, particularly, in a portion of the passage 15 extending along the reverse surface of the syringe base 11 in parallel with the diaphragm 12. The three electrodes 17 are used for real-time electrochemical examination of the substance (blood or body fluid) sampled through the needle 13, as described later.

Further, on the reserve side of the syringe base 11 is arranged a protective member 16 having an aperture 16a for passing the needle 13 therethrough. The protective member 16 faces the reverse surface of the syringe base 11 and is movable toward and away from the syringe base. A spring 19 is interposed between the protective member 16 and the reverse surface of the syringe base 11 such that the protective member 16 is elastically displaceable and, in a normal state, located more outward than the tip of the needle 13. The protective member 16 serves to prevent the needle 13 from accidentally touching (sticking) a person handling the sampling syringe unit 10. Especially, when the sampling syringe unit 10 is disposed of after it is used (blood or body fluid is sampled) in conjunction with a sampling device, described later, the protective member 16 prevents the handler from touching the needle 13.

Instead of using the spring, a magnet (not shown) may be attached to each of the reverse surface of the syringe base 11 and the inner surface of the protective member 16 facing the syringe base 11 such that in a normal state, the protective member 16 is located more outward than the tip of the needle 13 due to the repulsive force exerted by the magnets. Alternatively, the protective member 16 may be provided with a hook mechanism (not shown) for selectively engaging with an edge of the syringe base 11, and in a normal state, the hook mechanism may be engaged with the syringe base 11 to fix the position of the protective member 16 relative to the syringe base 11.

The sampling syringe unit 10 is removably attached to the sampling device when used, as mentioned above. For example, as shown in FIG. 1, the syringe base 11 is fitted at its edges into holders 21 of the sampling device 20 such that the protective member 16 (needle 13) faces outward.

The sampling device 20 basically comprises a first plunger (first actuator) 22 for pressing the syringe base 11 (needle 13) against the skin of a subject (patient). The sampling device 20 further includes a second plunger (second actuator) 23 for displacing the diaphragm 12 to reduce the capacity of the suction chamber defined by the recess 14 and then releasing the displacing force to restore the diaphragm 12 by its own elasticity and thereby cause a suction pressure to be produced in the suction chamber as the capacity thereof increases.

Specifically, the first plunger 22 has the function of pressing the syringe base 11 through, for example, the holders 21, against the subject's (patient's) skin and thereby causing the needle 13 protruding from the reverse surface of the syringe base 11 to be stuck into the subject's skin. Also, when the first plunger 22 releases the pressure and returns to its original state, it allows the needle 13 to be pulled out of the skin. The second plunger 23 elastically deforms the diaphragm 12 toward the inside of the recess 14, as shown in FIG. 2, to reduce the capacity of the chamber defined by the recess 14 (to expel air from the chamber), and then releases the deforming force to restore the diaphragm 12 by its own elasticity and thereby expand the chamber such that a negative pressure produced in the expanding chamber exerts a force of suction through the needle 13. In other words, the second plunger 23 serves as a pump whereby the chamber defined by the diaphragm 12 and the recess 14 functions as a suction chamber for exerting a suction force through the needle 13.

The sampling device 20 further comprises a controller 24 for actuating the plungers 22 and 23 at timings described later, in a manner associated with each other. The controller 24 includes a timer 25 for actuating the plungers 22 and 23 upon lapse of predetermined periods from the reception of commands for operating the plungers 22 and 23, respectively, such that blood or body fluid can be drawn into the sampling syringe unit 10 at desired timing set by the timer 25.

In the above sampling syringe unit, the suction chamber is defined by the recess 14 formed in the syringe base 11 and the flat diaphragm 12 covering the recess 14. Alternatively, the diaphragm 12 may be a hat-shaped elastic member made of synthetic resin, as shown in FIGS. 3A and 3B, for example, and a chamber 12a serving as the suction chamber may be defined by the recess of the diaphragm 12 and the flat surface of the syringe base 11.

The sampling device 20 is previously fitted with the sampling syringe unit 10 and is attached to the subject's arm or the like by using an adhesive tape, for example. As the first and second plungers 22 and 23 sequentially operate in association with each other under the control of the controller 24, the needle 13 is automatically stuck into the subject's (patient's) skin by the action of the first plunger 22 and the subject's (patient's) blood or body fluid is automatically sampled through the needle 13 by the action of the second plunger 23. Especially, by using the timer 25 to set the operation timings for the first and second plungers 22 and 23, it is possible to automatically sample blood or body fluid at desired timing.

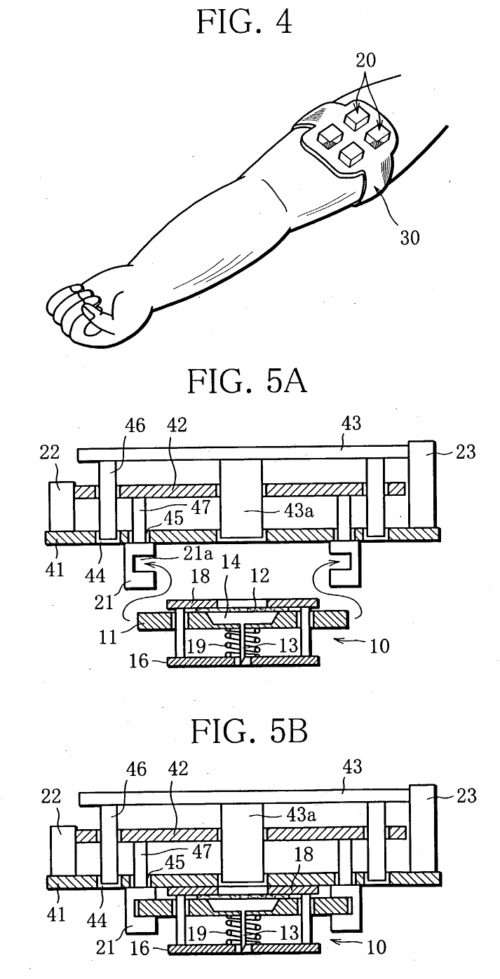

Also, a plurality of sampling devices 20 may be prepared and may be collectively attached to the subject's (patient's) arm by using an arm band 30, as shown in FIG. 4, for example. In this case, the operation timings for the sampling devices 20 may be set differently by the respective timers 25 so that the subject's (patient's) blood or body fluid can be automatically sampled at regular intervals of time, for example. Especially, where the electrodes 17 are used to electrochemically examine the sampled substance (blood or body fluid) in real time, in vivo change of the subject (patient) can be automatically monitored.

Although not illustrated, a suitable number of sampling devices 20 may be joined to constitute an array structure having the devices 20 arranged in the form of a 2×2 or 3×3 matrix, for example, and an equal number of sampling syringe units 10 to be attached to the respective sampling devices 20 may also be joined to constitute an array structure having the units 10 arranged at the same pitch as the sampling devices 20. With such array structures, it is possible to collectively attach and detach the multiple sampling syringe units 10, thus facilitating the handling.

To attach the sampling syringe unit 10 to the sampling device 20, the former may be affixed to the latter by using a Velcro (registered trademark) or an adhesive double coated tape, instead of inserting the syringe base 11 laterally into the grooves 21a of the holders 21 as mentioned above. Alternatively, magnetic powder may previously be admixed in the plastic material for making the sampling syringe unit 10 so that the sampling syringe unit 10 may be attracted to the sampling device 20 by magnetic attractive force.

The sampling device 20 will be now described in more detail. As schematically shown in FIG. 5A which exemplifies the functional arrangement of the sampling device 20, the sampling device 20 comprises a base plate 41 constituting a housing unit, and first and second movable plates 42 and 43 arranged over the base plate 41. The first and second movable plates 42 and 43 are supported by guide pins 47 and 46, respectively, which are inserted through respective guide holes 45 and 44 formed in the base plate 41, and are movable toward and away from the base plate 41 while being kept parallel thereto. The first and second movable plates 42 and 43 are actuated by first and second plungers 22 and 23, respectively, which are mounted to the base plate (housing unit) 41.

The plungers 22 and 23 are each a reciprocating type of which the actuator can be driven in opposite directions by changing the polarity of driving current. Alternatively, each of the plungers 22 and 23 may be a self-returning type whose actuator is urged in one direction by a spring (not shown), moves in the opposite direction against the urging force of the spring when energized, and automatically returns to the original position when de-energized.

The holders 21, to which the sampling syringe unit 10 is removably attached, are formed at distal ends of the respective guide pins 45, for example, to hold the sampling syringe unit 10 on the reverse surface (lower surface) of the base plate 41. These two holders 21 have grooves 21a, respectively, facing each other and extending along the reverse surface of the base plate 41. The sampling syringe unit 10 is attached to the sampling device 20 by inserting edges of the syringe base 11 into the respective grooves 21a from one side of the sampling device 20.

The sampling syringe unit 10 shown in FIG. 5A includes, in addition to the basic elements described above, a flat plate-like auxiliary protective member 18 arranged on the surface of the syringe base 11. The primary function of the auxiliary protective member 18 is to prevent a finger etc. of a person handling the sampling syringe unit 10 from directly touching the diaphragm 12. Further, the auxiliary protective member 18 serves to stabilize the orientation of the sampling syringe unit 10 relative to the sampling device 20, as well as to increase the strength of supports connecting the auxiliary protective member 18 and the protective member 16. Also, the auxiliary protective member 18 has a hole formed in a central portion thereof, and the hole has a size permitting passage of a pusher pin 43a protruding from the second movable plate 43. The auxiliary protective member 18 is connected by the supports to the protective member 16 with the syringe base 11 located therebetween, and thus is displaceable together with the protective member 16 relative to the syringe base 11.

When the sampling syringe unit 10 constructed as above is attached to or detached from the sampling device 20, the first movable plate 42 is preferably lowered by temporarily operating the first plunger 22 or by manually applying a downward force, for example, to cause the holders 21 to project farther from the lower surface of the base plate 41, thereby securing a sufficient working space between the holders 21 under the reverse surface (lower surface) of the base plate 41. This state of the sampling device makes it easier to attach or detach the sampling syringe unit 10 to or from the holders 21. After the sampling syringe unit 10 is fitted to the holders 21, the holders 21 are returned to their original position by stopping the operation of the first plunger 22 or by releasing the first movable plate 42 from the downward force, whereupon the sampling syringe unit 10 is securely held between the holders 21 and the lower surface of the base plate 41, as shown in FIG. 5B.

In this case, the auxiliary protective member 18 is interposed between the syringe base 11 held by the holders 21 and the lower surface of the base plate 41, whereby the orientation of the sampling syringe unit is stabilized. The protective member 16 connected to the auxiliary protective member 18 is fixed at a location more downward (outward) than the tip of the needle 13, as shown in FIG. 5B. Consequently, the protective member 16 protects the needle 13 while keeping the needle 13 situated more inward than the protective member 16. Also, the protective member 16 prevents a person handling the sampling device 20 from touching the needle 13 or the needle 13 from accidentally sticking the subject (patient) to whom the sampling device 20 is attached.

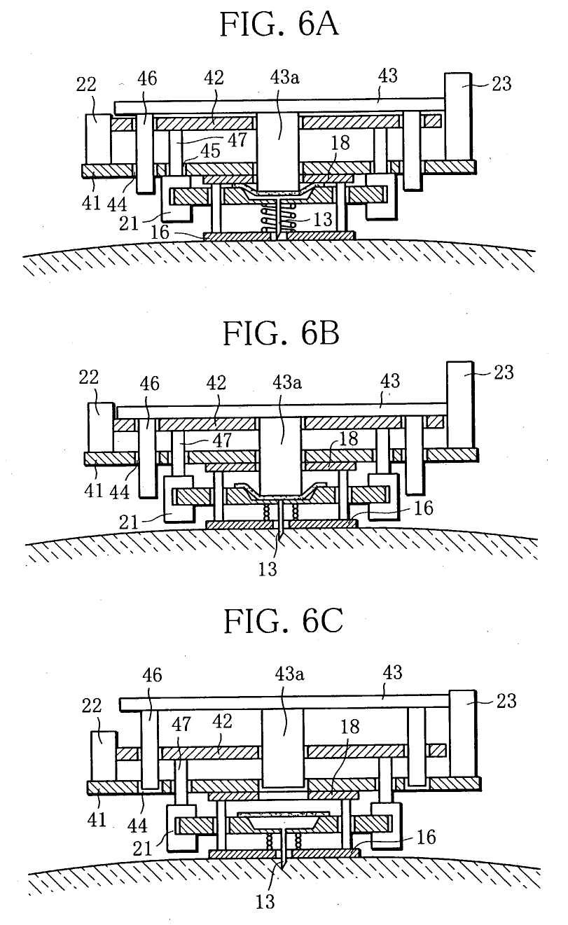

The sampling device 20 thus fitted with the sampling syringe unit 10 is affixed to the arm or the like of the subject (patient), and with the protective member 16 kept in contact with the skin, the sampling device 20 is operated as described below, under the control of the controller 24. First, the second plunger 23 is operated to lower the second movable plate 43 toward the base plate 41, as shown in FIG. 6A, thereby pressing the diaphragm 12 with the pusher pin 43a provided on the second movable plate 43. Consequently, the diaphragm 12 is elastically deformed, with the result that the capacity of the suction chamber defined by the recess 14 decreases.

Subsequently, the first plunger 22 is operated to lower the first movable plate 42 toward the base plate 41, thereby lowering the syringe base 11 through the holders 21 toward the skin. At this time, the second plunger 22 is kept operating, so that the second movable plate 43 is further moved toward the base plate 41 with displacement of the syringe base 11. As a result, the elastically deformed state of the diaphragm 12 pressed by the pusher pin 43a is maintained. Since the protective member 16 connected to the auxiliary protective member 18 remains in contact with the skin and movement thereof is obstructed, the syringe base 11 alone is moved toward the skin by the action of the first plunger 22, as shown in FIG. 6B, and the needle 13 protruding from the reverse surface of the syringe base 11 is stuck into the skin.

While in this state, the operation of the second plunger 23 is stopped and the second movable plate 43 is returned to the original position, as shown in FIG. 6C, whereupon the diaphragm 12 is released from the elastic deformation and thus is restored to its original state due to its own elasticity. As the diaphragm 12 is elastically restored, the capacity of the suction chamber increases to produce a suction pressure therein, causing the needle 13 stuck into the skin to exert a suction force. Due to the suction force, the blood or body fluid under the skin is drawn through the needle 13 into the communication passage 15 and then into the recess 14.

Subsequently, the operation of the first plunger 22 is stopped, whereupon the first movable plate 42 returns to the original position and thus the syringe base 11 is raised, so that the needle 13 is retracted to the inside of the protective member 16, as shown in FIG. 5B, and thus is pulled out of the skin.

The aforementioned sequence of control operations for the first and second plungers 22 and 23 makes it possible to automatically carry out the elastic deformation of the diaphragm 12 as a preparatory step, the insertion of the needle 13 into the skin, the sampling of blood or body fluid through the needle 13 by the elastic restoration of the diaphragm 12 (suction into the suction chamber), and the removal of the needle 13 from the skin.

The sampling device 20 can be constructed by arranging the base plate 41, the first movable plate 42 and the second movable plate 43, each made of a thin plate, one over another such that the thin plates are displaceable in a direction perpendicular to their surfaces over a range of about 1 to 2 mm necessary to stick the needle 13 into the skin, and accordingly, the sampling device 20 can be easily reduced in thickness. Also, the sampling syringe unit 10 attached to the sampling device 20 can be easily reduced in thickness because the syringe base 11 has only to be displaced in a direction perpendicular to the surface of the diaphragm 12 (in the axial direction of the needle 13) to move the needle 13 protruding in a direction substantially perpendicular to the surface of the diaphragm 12. Further, the direction of displacement of the diaphragm 12 can be made identical with the direction of displacement of the syringe base 11. Consequently, the sampling device 20 fitted with the sampling syringe unit 10 can be reduced in thickness and in size as a whole, and thus can be easily attached to the arm or the like of the subject (patient).

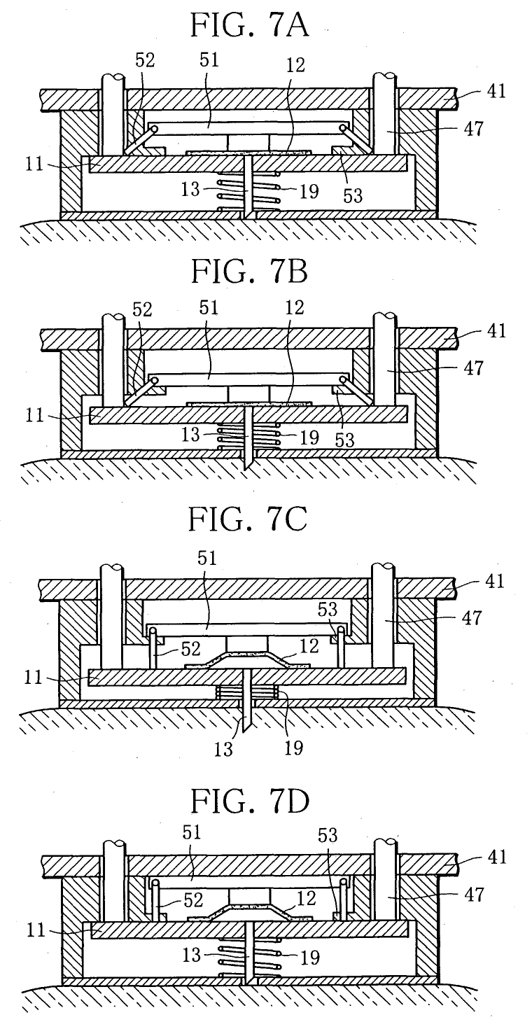

The sampling device 20 described above is provided with the two plungers 22 and 23 so that the syringe base 11 and the diaphragm 12 may be displaced in association with each other. Alternatively, such displacing operations may be carried out by a single plunger. In this case, the sampling syringe unit 10 may be constructed as shown in FIGS. 7A to 7D, for example.

Specifically, only the outer peripheral edge portion of a flat diaphragm 12 is bonded to the upper surface of a flat plate-like syringe base 11 so that, when the central portion of the diaphragm 12 is pulled up by elastic deformation, a chamber serving as the suction chamber may be defined between the diaphragm 12 and the upper surface of the syringe base 11. Also, a flat plate-like auxiliary member 51 for pulling up the diaphragm 12 is arranged parallel to the diaphragm 12 at a predetermined distance from the upper surface of the syringe base 11. Further, stoppers 52 are pivotally connected to the auxiliary member 51 so that, when the diaphragm 12 is elastically deformed, the stoppers 52 may hang down and intervene between the upper surface of the syringe base 11 and the auxiliary member 51 to maintain the elastically deformed state of the diaphragm 12.

The stoppers 52 may each be a pivotable lever that turns by its own weight to intervene between the upper surface of the syringe base 11 and the auxiliary member 51 when the interval between the syringe base 11 and the auxiliary member 51 is expanded and thus the diaphragm 12 is elastically deformed. Alternatively, each stopper 52 may be an elastic piece which is elastically deformed in advance between the syringe base 11 and the auxiliary member 51. When the interval between the syringe base 11 and the auxiliary member 51 is expanded and thus the diaphragm 12 is elastically deformed, the elastic pieces as the stoppers 52 are restored to the original state due to their own elasticity to fix the expanded interval between the syringe base 11 and the auxiliary member 51.

In the case of using the sampling syringe unit 10 having the auxiliary member 51 and the stoppers 52, the sampling device 20 is provided with engaging portions 53, as shown in FIG. 7A, for example, for limiting the amount of displacement of the auxiliary member 51 and thereby pulling up the diaphragm 12. The sampling syringe unit 10 is attached to the lower surface of the sampling device 20 such that the auxiliary member 51 is located between the engaging portions 53 under the base plate 41 constituting the housing unit.

With the sampling syringe unit 10 attached to the sampling device 20, a plunger (not shown) incorporated in the sampling device is driven to lower its actuator, whereupon the syringe base 11 is displaced in a direction toward the lower surface thereof on which the needle 13 is provided, as shown in FIG. 7B, and the auxiliary member 51 also moves downward with displacement of the syringe base 11 until it abuts against the engaging portions 53. As the syringe base 11 is displaced, the needle 13 protruding from the lower surface of the syringe base 11 is stuck into the subject's (patient's) skin.

As the actuator is further lowered thereafter, the needle 13 is stuck deeper into the skin, as shown in FIG. 7C, while the auxiliary member 51 abuts against the engaging portions 53 and thus further displacement (downward movement) thereof is obstructed. Consequently, the central portion of the diaphragm 12 joined to the auxiliary member 51 is pulled up, creating the suction chamber between the syringe base 11 and the elastically deformed diaphragm 12. Due to the resulting suction pressure produced in the chamber, blood or body fluid is sampled from the subject (patient) through the needle 13. At this time, as the interval between the auxiliary member 51 and the syringe base 11 expands, the stoppers 52 connected to the auxiliary member 51 turn downward due to their own weight and intervene between the syringe base 11 and the auxiliary member 51, to maintain the expanded interval between the auxiliary member 51 and the syringe base 11. Consequently, the elastically deformed state of the diaphragm 12 defining the suction chamber therein is maintained.

Subsequently, the plunger is driven in the opposite direction to raise the syringe base 11, whereby the needle 13 is pulled out of the subject's (patient's) skin, as shown in FIG. 7D. In this case, since the deformed state of the diaphragm 12 is maintained by the stoppers 52, the blood or the like in the chamber defined between the diaphragm 12 and the syringe base 11 does not leak out through the needle 13.

Like the foregoing embodiment, the sampling syringe unit 10 and the sampling device 20 constructed in this manner permit automatic sampling of blood or body fluid from the subject (patient). Especially, in this embodiment, the sampling device 20 has only one plunger (actuator) incorporated therein, and accordingly, the sampling device can be simplified in structure, compared with the foregoing embodiment. Further, reciprocating motion of the single plunger (actuator) makes it possible to sequentially carry out the insertion of the needle 13 into the skin, the sampling of blood or body fluid by deformation of the diaphragm 12, and the removal of the needle 13 in a manner associated with one another, whereby the operation reliability of the sampling device can be significantly enhanced.

The quantity of blood or body fluid to be sampled by the sampling syringe unit 10 may be about 10 μl at most. Accordingly, the syringe base 11 itself can be easily formed as a small-sized rectangular chip having sides of about 4 to 5 mm long and a thickness of about 2 mm, for example. Also, the needle 13 with an outside diameter of 0.1 mm or less can be easily obtained by micromachining a stainless steel pipe. Such a minute needle 13 is thinner than the average intervals at which pain spots are situated on the human skin, and in most cases, therefore, it is possible to stick the needle 13 in regions clear of pain spots. Consequently, blood or body fluid can be sampled from the subject (patient) without giving him/her a disagreeable sensation such as pain.

Also, when the sampling syringe unit 10 with the aforementioned structure is detached from the sampling device 20 after use, the needle 13 is covered with the protective member 16 and does not protrude to outside. It is therefore possible to prevent a person handling the sampling syringe unit from touching the needle 13 by accident, thus ensuring hygienic safety. Further, since the diaphragm 12 is also covered with the auxiliary protective member 18, it is possible to effectively prevent the handler from accidentally pressing the diaphragm 12, and thus to prevent the sampled blood or body fluid from leaking out of the sampling syringe unit 10. The sampling syringe unit can therefore satisfactorily function as a disposable type medical instrument designed to prevent infection.

Further, the sampling device 20 can be operated at desired timing by using the timer 25, as mentioned above. Thus, if multiple sampling devices 20 are attached to the subject (patient) by using the arm band 30, as shown in FIG. 4, and are automatically operated thereafter at given intervals of time, then blood or body fluid can be sampled from the subject (patient) at regular intervals. It is therefore possible to automatically sample blood or body fluid a plurality of times with ease.

The present invention is not limited to the above embodiments alone. For example, a sensing circuit for measuring the blood sugar level etc. may be packaged into the sampling device 20. Also, in cases where the sampled substance is electrochemically examined in real time by means of the electrodes 17, the blood or body fluid need not be drawn up to the recess 14, and accordingly, the suction force (negative pressure) in the suction chamber defined by the recess 14 may be reduced to a lower level. In this case, the structure of the sampling syringe unit 10 can be further simplified with ease. It is to be noted that the present invention can be modified in various other ways without departing from the spirit and scope of the invention.

As described above, the present invention provides a small-sized sampling syringe unit which can be easily reduced in thickness and is easy to handle, and a sampling device to which the sampling syringe unit is removably attached when used. The present invention is advantageous in that the sampling device can be operated at desired timing. Also, the present invention provides a highly practical sampling method using the sampling device and capable of effectively carrying out automatic sampling of blood or body fluid from a subject.

1. A sampling syringe unit for sampling blood or body fluid, comprising:
a syringe base;
a diaphragm attached to one surface of said syringe base, for defining a chamber in cooperation with said syringe base;
a hollow tubular needle protruding substantially perpendicularly from a surface of said syringe base opposite to the surface on which said diaphragm is arranged, and communicating with the chamber; and
a protective member having an aperture for passing said needle therethrough, said protective member facing the surface of said syringe base from which said needle protrudes and movable toward and away from said syringe base, wherein in a normal state, said protective member is located more outward than a tip of said needle.

2. The sampling syringe unit according to claim 1, wherein said sampling syringe unit is a disposable type removably attached to a sampling device.

3. The sampling syringe unit according to claim 1, wherein said needle protrudes from a central portion of said syringe base and has an outer diameter of 0.1 mm or less.

4. The sampling syringe unit according to claim 1, wherein said syringe base has electrodes arranged in a communication passage connecting the chamber and said needle for examining a substance sampled through said needle.

5. The sampling syringe unit according to claim 4, wherein said electrodes are arranged in a portion of the communication passage extending parallel with said diaphragm.

6. A sampling device for sampling blood or body fluid, comprising:
a housing unit to which the sampling syringe unit according to claim 1 is removably attached in such a manner that the surface of the syringe base on which the needle is arranged faces outward;
a first actuator arranged in said housing unit, for displacing the syringe base of the sampling syringe unit attached to said housing unit in a direction toward the surface of the syringe base on which the needle is arranged; and
a second actuator arranged in said housing unit and driven in association with said first actuator, for displacing the diaphragm in a direction toward the surface of the syringe base on which the needle is arranged, and then releasing the displacing force to restore the diaphragm and thereby cause a suction pressure to be produced in the chamber.

7. A sampling device for sampling blood or body fluid, comprising:
a housing unit to which the sampling syringe unit according to claim 1 is removably attached in such a manner that the surface of the syringe base on which the needle is arranged faces outward;
an actuator arranged in said housing unit, for displacing the syringe base of the sampling syringe unit attached to said housing unit in a direction toward the surface of the syringe base on which the needle is arranged; and
a deformation control member for deforming the diaphragm as the syringe base is displaced, to cause a suction pressure to be produced in the chamber, and then maintaining a deformed state of the diaphragm.

8. The sampling device according to claim 6 or 7, further comprising a controller incorporated in said housing unit for controlling operation timing of said actuators/actuator.

9. A method of sampling blood or body fluid, comprising:
attaching the sampling devices according to claim 8 to a subject; and
sequentially driving the sampling devices to sample blood or body fluid from the subject at different times.
